(19) United States
(12) Patent Application Publication
(10) Pub. No.: US 2010/0069483 A1
(43) Pub. Date: Mar. 18, 2010

(54) DUAL INHIBITION OF CYCLOOXYGENASE-2 AND CARBONIC ANHYDRASE

(75) Inventors: Mi-Young PARK, Anyang-si (KR); Kyung-Min LIM, Suwon-si (KR); Song-Seok SHIN, Youngin-si (KR); Jun-Yong HA, Seoul (KR); Shin CHUNG, Yongin-si (KR)

Correspondence Address: SUGHRUE MION, PLLC, 2100 PENNSYLVANIA AVENUE, N.W., SUITE 800, WASHINGTON, DC, 20037, US

(73) Assignee: AMOREPACIFIC CORPORATION, Seoul (KR)

(21) Appl. No.: 12/623,971

(22) Filed: Nov. 23, 2009

Related U.S. Application Data
(62) Division of application No. 11/091,376, filed on Mar. 29, 2005.
(60) Provisional application No. 60/557,508, filed on Mar. 30, 2004.
(60) Provisional application No. 60/611,728, filed on Sep. 21, 2004.

(51) Int. Cl. A61K 31/34

(52) U.S. Cl. 514/471

(57) ABSTRACT

Compounds of Formula I potently inhibit both cyclooxygenase-2 and carbonic anhydrases. Inhibition of carbonic anhydrases by a cyclooxygenase-2 inhibitor may affect significantly the safety and efficacy profiles of such a dual inhibitor in the treatment of cyclooxygenase-2 mediated disorders, compared to a cyclooxygenase-2 inhibitor without carbonic anhydrase inhibitory activity.

### CROSS REFERENCE TO RELATED APPLICATION

This is a Divisional of U.S. application Ser. No. 11/091,376, which claims benefit under 35 U.S.C. §119(e) of U.S. provisional application Ser. Nos. 60/557,508 filed on Mar. 30, 2004 and 60/611,728 filed on Sep. 21, 2004, the disclosures of which are hereby incorporated by reference in their entirety.

### FIELD OF INVENTION

The present invention relates to a class of compounds that potently inhibit both cyclooxygenase-2 and carbonic anhydrase(s). Also the present invention relates to clinical benefits in the treatment or prevention of cyclooxygenase-2 mediated disorders by concomitant inhibition of cyclooxygenase-2 and carbonic anhydrase(s).

### BACKGROUND OF THE INVENTION

Cyclooxygenases are enzymes involved in the transformation of arachidonic acid into a variety of prostaglandins and thromboxanes. To date, there are at least two kinds of cyclooxygenases discovered. Cyclooxygenase-1 (COX-1) is constitutively expressed in a variety of tissues including the gastro-intestinal (GI) mucosa and the kidney. COX-1 is believed to be responsible for the maintenance of the homeostasis, for example, in the GI tract Inhibition of COX-1 is known to be associated with the undesirable toxicity of perforation, ulceration and bleeding in the GI tract. In the meantime, cyclooxygenase-2 (COX-2) is induced upon inflammatory stimuli and known to be involved in the pathogenesis of inflammation and inflammation-associated disorders. Physiological and clinical aspects of COX-2 inhibitors have been reviewed from diverse perspectives. It has to be noted that the therapeutic scope of COX-2 inhibitors may comprise not only inflammation and inflammation-associated disorders, but also other COX-2 mediated disorders including but not limited to cancers and Alzheimer's disease. [Expert Opin. Ther. Patents vol 15, 9-32 (2005); Pharmacol. Rev. vol 56, 387-437 (2004); Nature Rev. Drug Discovery vol 2, 879-890 (2003)]

Traditional non-steroidal anti-inflammatory drugs (NSAIDs), such as aspirin, naproxen, ibuprofen, piroxicam, diclofenac, and so on, inhibit both COX-1 and COX-2 at therapeutically relevant exposure. Even though traditional NSAIDs have been widely used over a century to treat inflammation and inflammation-associated disorders, the notorious life threatening GI toxicity of traditional NSAIDs has posed big concerns in the use of traditional NSAIDs for the treatment of osteoarthritis, rheumatoid arthritis, gouty arthritis, low back pain, migraines, post-operative pains, cancer pain, menstrual pain, ankylosing spondylitis, tendinitis, dental pain, and so on.

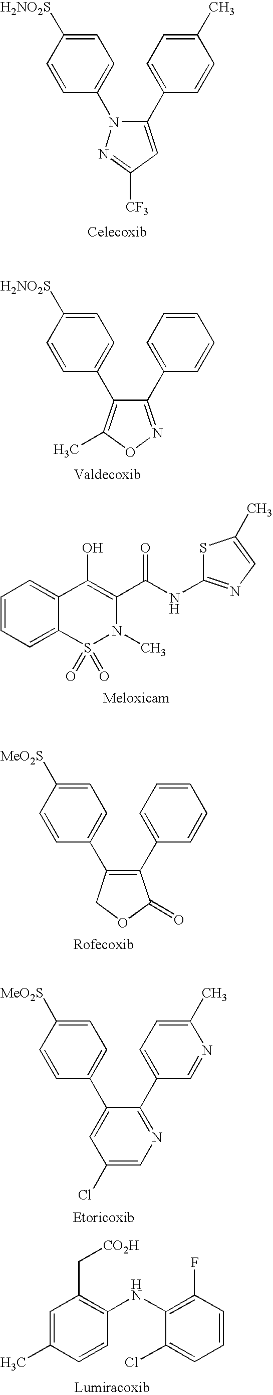

In order to reduce the notorious GI toxicity from inhibition of COX-1, selective COX-2 inhibitors have been extensively studied. To date, various selective COX-2 inhibitors, are now available for clinical use, which include celecoxib, rofecoxib, valdecoxib, etoricoxib, lumiracoxib, and meloxicam. Clinical evaluation with selective COX-2 inhibitors clearly demonstrated improved GI safety compared to traditional NSAIDs. [N. Engl. J. Med. vol 343, 1520-1528 (2000); JAMA vol 284, 1247-1255 (2000); Lancet vol 364, 675-684 (2004)] Even though selective COX-2 inhibitors are regarded superior to traditional NSAIDs in the GI safety, the GI adverse events from use of selective COX-2 inhibitors may become more pronounced in susceptible populations such as elderly patients and patients on anti-tumor therapy. [EJC Suppl. vol 2, 14-20 (2004); JAMA vol 289, 2816-2819 (2003)] Furthermore, in vivo findings suggest that COX-2 upregulation plays a crucial role in adaptive cytoprotection against mild irritation in the stomach. [J. Clin. Enterol. vol 25, S105-S110 (1997); Br. J. Pharmacol. vol 123, 927-935 (1998)] Other findings suggest that COX-2 may be important for the wound healing in the GI tract. [J. Clin. Enterol. vol 27, S28-S34 (1998)] Thus, chronic inhibition of the COX-2 in the GI tract could lead to unwanted adverse events in the GI tract. COX-2 needs to be inhibited for anti-inflammatory and analgesic effect, though. It is desired to minimize the inhibition of the COX-2 in the GI tract for the GI safety.

In rats, the jejunum and ileum were the primary target organs of the GI adverse events following repeated oral administrations of rofecoxib and celecoxib. Similar situations were observed in repeat oral dosing studies with valdecoxib. [Pharmacology Review (Rofecoxib), US FDA Application #21-042, pp 72-92; Pharmacology Review (Celecoxib), US FDA Application #20-998, pp 10-42; Pharmacology Review (Valdecoxib), US FDA Application #21-341, pp 27-54] Even though selective COX-2 inhibitors are considered to possess better GI safety than traditional NSAIDs, not all selective COX-2 inhibitors appear to show the same pattern of GI toxicity. Meloxicam is a selective COX-2 inhibitor of a modest COX-2 selectivity over COX-1 (13-fold selectivity in human whole blood). Repeat dosing of meloxicam in rats resulted in adverse findings in the stomach and the intestines. However, gastric findings such as peptic pyloric ulcers started coming out at lower dose than intestinal findings of duodenal perforations with peritonitis. [Pharmacology Review (Meloxicam), US FDA Application #20-938, pp 25-43] Such observation could be explained by a significant extent of COX-1 inhibition in the stomach, paralleling with the GI toxicity pattern of traditional NSAIDs. [Gut vol 49, 443-453 (2001)] The duodenum was the primary target site of the intestinal adverse events following repeat dosing of meloxicam in rats.

The most frequent renal adverse events of selective COX-2 inhibitors are edema, sodium retention, and the resultant hypertension. Given that selective COX-2 inhibitors and traditional NSAIDs are taken similar with regards to the renal safety, renal adverse effects are believed to originate much from the inhibition of the COX-2 expressed in the kidney. [J. Pain Symptom Management vol 23, S15-S20 (2002); J. Pharmacol. Exp. Therapeut. vol 289, 735-741 (2001)] Expression of the renal COX-2 was reported to increase with age, which could explain higher renal susceptibility in older people to the use of traditional NSAIDs and selective COX-2 inhibitors. [Kidney International vol 65, 510-520 (2004)] It is often the case that renal adverse events are a frequent cause of dropout for COX-2 inhibitors or traditional NSAIDs.

Analyses of clinical data with selective COX-2 inhibitors suggested that use of selective COX-2 inhibitors might be associated with an increase in the cardiotoxicity compared to use of a traditional NSAIDs. [JAMA vol 286, 954-959 (2001)] Unlike traditional NSAIDs, selective COX-2 inhibitors lack the anti-thrombotic effect from the COX-1 inhibition in the platelet, which could account for a potential increase in the cardiotoxicty by use of a selective COX-2 inhibitor. Recently, rofecoxib 25 mg/day was found to be associated with an increased risk of thromboembolic cardiovascular events in a long term cancer prevention trial (APPROVe). Very recently celecoxib at 400 mg/day and 800 mg/day was associated with increases in the thromboembolic events in a long term cancer prevention trial (APC). Further, acute use of valdecoxib was found to be associated with an increase in the thromboembolic events in coronary artery bypass graft patients. [Biocentury vol 13, No 4, A1-A4 (2005)]

Given that hypertension is a well-established risk factor for thromboembolic events, [Lancet vol 335, 827-838 (1990)] improvement in the renal safety would be useful to reduce the potential cardiotoxicity of a selective COX-2 inhibitor. On the other hand, the COX-2 expressed in the endothelial layer (i.e. endothelial COX-2) produces vasodilatory prostacyclin (prostaglandin I₂), which was shown to inhibit the prothrombotic activity of the platelet. Thus, a reduction in the circulatory level of prostacyclin could increase the risk of hypertension as well as thromboembolic events. [Science vol 296, 539-541 (2002)] The observed increase in the cardiotoxicity from the acute use of valdecoxib could be better explained by the inhibition of the endothelial COX-2 than by the inhibition of the renal COX-2. [Biocentury vol 13, No 4, A1-A4 (2005)] Nevertheless, inhibition of the renal COX-2 would have contributed significantly to the observed cardiotoxicity of rofecoxib and celecoxib in the long term clinical trials. [Cleveland Clinic J. Med. vol 71 849-856 (2004)] COX-2 needs to be inhibited for the anti-inflammatory and analgesic effect in tissues of therapeutic concern, however, the inhibition of COX-2 in the systemic circulation and kidney should be minimized for the renal and cardiovascular safety.

Carbonic anhydrases (CAs, EC 4.2.1.1) are wide-spread zinc-containing enzymes, which catalyze the hydration of carbon dioxide (CO₂+H₂O [symbol] H₂CO₃ [symbol] H⁺+HCO₃⁻). To date, at least 14 isozymes of CAs have been discovered. CAs are present as either cytosolic or membrane bound form. For example, CA I and CA II are cytosolic enzymes, whilst CA IV and CA IX are membrane-bound. Physiological roles of CAs have been studied over decades. Supuran and Scozzafava published an excellent review article on CA inhibitors from diverse perspectives. [Expert Opin. Ther. Patents vol 10, 575-600 (2000)]

Inhibitors of CAs have been found effective in treating a variety of CA mediated disorders, including but not limited to glaucoma, macular edema, hydrocephalus, high altitude disease (mountain sickness), upper GI ulcers, some types of cancers, and so on. CA inhibitors have been found useful as diuretics to treat patients with edema and congested heart failure Inhibition of the CAs, especially CA II, expressed in the kidney is believed to be responsible for the diuretic activity of CA inhibitors Inhibition of CAs in the upper GI tract increases the secretion of bicarbonate or decreases the secretion of the gastric acid, which would be useful to counteract to overt presence of gastric acid in the upper GI tract. Given that CA II is abundantly present in the osteoclast, inhibition of CA II may be useful to suppress osteoclastogenesis by decreasing H⁺ release from the osteoclast.

Many of CA inhibitors are aromatic sulfonamide derivatives. Sulfonamide CA inhibitors have a solid position mainly in the treatment of glaucoma, fluid retention and some neurological disorders. It is well established that aromatic sulfonamide moiety strongly coordinates to the zinc ion in CA. Acetazolamide, methazolamide, ethoxolamide, dichlorophenamide, dorozolamide and brinzolamide are aromatic sulfonamide CA inhibitors, which have been in clinical use. Nevertheless, it needs to be noted that overt inhibition of CAs through systemic use of a non-selective CA inhibitor could be associated with undesirable side effects including metabolic acidosis, electrolyte imbalance, fatigue, gastrointestinal irritation and hyper or hypoglycemia. Therefore it is desired for safety reasons not to overtly inhibit CAs in the whole body. Due to their abundant presence, however, CAs are hardly overtly occupied by administration of a carbonic anhydrase inhibitor at small doses regardless of its CA inhibitory potencies.

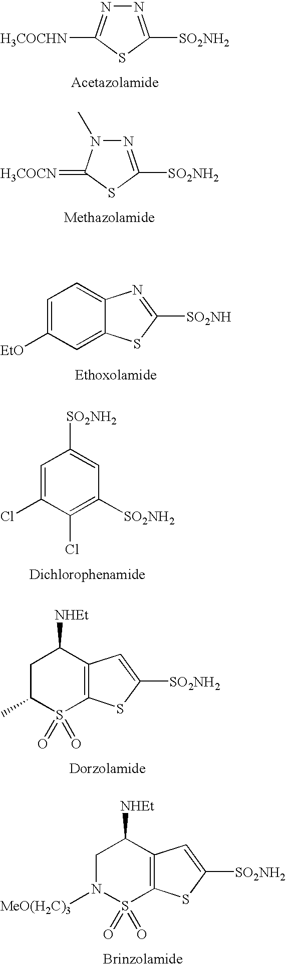

Prior arts WO 2004/014352, US Patent Application 2003/0220376, and WO 03/013655 provided phenylsulfonamide derivatives including celecoxib and valdecoxib as dual inhibitors of CAs and COX/COX-2, and methods to treat or prevent disorders mediated through CA(s) by administration of such a dual inhibitor to a subject. According to Supuran and coworkers, oral administration of celecoxib or valdecoxib reduced intraocular pressure in hypertensive rabbits, whereas NSAID diclofenac failed to reduce the intraocular pressure. [J. Med. Chem. vol 47, 550-557 (2004)] The observed effect by celecoxib and valdecoxib was ascribed to the inhibition of CAs rather than COX-2.

Prior art WO 00/61571 provided a novel class of COX-2 inhibitors represented by Formula A with 3(2H)furanone as a scaffold or pharmacophore for potent selective inhibition of COX-2 over COX-1,

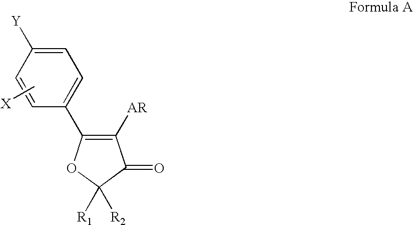

wherein:
X represents halo, hydrido, or alkyl;
Y represents alkylsulfonyl, aminosulfonyl, alkylsulfinyl, (N-acylamino)-sulfonyl, (N-alkylamino)sulfonyl, or alkylthio;
R₁ and R₂ are selected independently from lower alkyl radicals, or form a 4- to 6-membered aliphatic or heterocyclic group, taken together with the 2-position carbon atom of 3(2H)-furanone ring; and
AR represents a substituted or non-substituted aromatic group of 5 to 10 skeletal atoms.

Compounds of Formula A are selective COX-2 inhibitors with strong anti-inflammatory and analgesic activities in animal models, as demonstrated in the prior art WO 00/61571 and the literature. [J. Med. Chem. vol 47, 792-804 (2004)] For example, 5-{4-(aminosulfonyl)phenyl}-2,2-dimethyl-4-(3-fluorophenyl)-3(2H)furanone (Example 1 of this invention) showed an ED₅₀ of 0.1 mg/kg/day by adjuvant-induced arthritis in male Lewis rats, whereas an ED₅₀ of 0.3 mg/kg/day was observed with positive comparator indomethacin. In carrageenan-induced thermal hyperalgesia in male SD rats, ED₅₀'s of 0.25 mg/kg and ˜1.0 mg/kg were observed for orally administered Example 1 and indomethacin, respectively. Given that adjuvant-induced arthritis simulates well the pathogenic situations of human arthritis, a selective COX-2 inhibitor showing a strong potency by adjuvant-induced arthritis would show therapeutic activity at a small daily dose for the treatment of osteoarthritis and rheumatoid arthritis.

### SUMMARY OF THE INVENTION

This invention provides several embodiments relating to the inhibition of CAs by a compound of Formula I, and the clinical benefits in the treatment or prevention of disorders mediated through COX-2 or carbonic anhydrases by administering to a subject a compound of Formula I, or a pharmaceutically acceptable salt or composition thereof

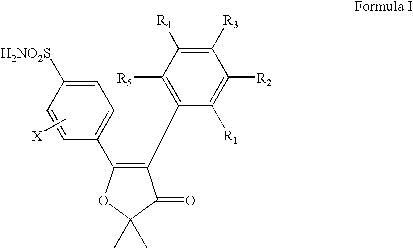

In one embodiment of the present invention, compounds of Formula I are demonstrated to potently inhibit CAs:

wherein,
X is selected from halo, hydrido, or lower alkyl; and
each of R₁ to R₅, if present, is selected independently from hydrido, halo, alkyl, haloalkyl, acyl, alkoxy, hydroxy, nitro, amino, N-alkylamino, N-acylamino, cyano, formyl, or azido; or two adjacent groups of R₁ to R₅ form, taken together, methylenedioxy.

A compound of Formula I can be converted into a pharmaceutically-acceptable salt by neutralizing the compound, depending on the presence of an acidic group or a basic group in the compound, with an equivalent amount of an appropriate pharmaceutically-acceptable acid or base, such as potassium hydroxide, sodium hydroxide, hydrochloric acid, methansulfonic acid, citric acid, and the like. A compound of Formula I or a pharmaceutically-acceptable salt thereof can be administered along with various pharmaceutically-acceptable adjuvant ingredients, including but not limited to, citric acid, sodium chloride, tartaric acid, stearic acid, starch, gelatin, talc, sesame oil, ascrobic acid, methylcellulose, sodium carboxymethylcelluose, polyethyleneglycol (PEG), polypropyleneglycol, sweeteners, preservatives, water, ethanol, titanium oxide, sodium bicarbonate, silicified microcrystalline cellulose, soybean lecithin, and the like. A compound of Formula I or a pharmaceutically-acceptable salt thereof can be formulated in a variety of dosage forms, including but not limited to, tablet, powder, granule, hard capsule, soft capsule, oral suspension, spray solution for inhalation, injectable solution, cream for topical application, transdermal patch, and the like. A compound of Formula I or a pharmaceutically-acceptable salt thereof can be administered to a human or animal subject at a daily dose of up to 100 mg/kg body weight but preferably up to 10 mg/kg body weight, depending on the indications, symptoms, or conditions of the subject.

In another embodiment, a method is provided to reduce the toxicity associated with COX-2 inhibition in the treatment or prevention of COX-2 mediated disorders through COX-2 inhibition by administering to a subject a therapeutically relevant amount of a compound of Formula I, or a pharmaceutically acceptable salt or composition thereof:

wherein,
X is selected from halo, hydrido, or lower alkyl; and
each of R₁ to R₅, if present, is selected independently from hydrido, halo, alkyl, haloalkyl, acyl, alkoxy, hydroxy, nitro, amino, N-alkylamino, N-acylamino, cyano, formyl, or azido; or two adjacent groups of R₁ to R₅ form, taken together, methylenedioxy.

In yet another embodiment, a method is provided to improve the therapeutic efficacy in the treatment or prevention of disorders mediated by COX-2 and carbonic anhydrases by administering to a subject a compound of Formula I, or a pharmaceutically acceptable salt or composition thereof, compared to the therapeutic efficacy by inhibition of either COX-2 or carbonic anhydrases alone:

wherein,
X is selected from halo, hydrido, or lower alkyl; and
each of R₁ to R₅, if present, is selected independently from hydrido, halo, alkyl, haloalkyl, acyl, alkoxy, hydroxy, nitro, amino, N-alkylamino, N-acylamino, cyano, formyl, or azido; or two adjacent groups of R₁ to R₅ form, taken together, methylenedioxy.

In a preferred embodiment of the present invention, interested compounds of Formula I are demonstrated to potently inhibit CAs:

wherein,
X is selected from fluoro, chloro, hydrido, or methyl; and
each of R₁ to R₅, if present, is selected independently from hydrido or halo.

In another preferred embodiment, a method is provided to reduce the toxicity associated with COX-2 inhibition in the treatment or prevention of COX-2 mediated disorders through COX-2 inhibition by administering to a subject a therapeutically relevant amount of an interested compound of Formula I, or a pharmaceutically acceptable salt or composition thereof:

wherein,
X is selected from fluoro, chloro, hydrido, or methyl; and
each of R₁ to R₅, if present, is selected independently from hydrido, or halo.

In yet another preferred embodiment, a method is provided to improve the therapeutic efficacy in the treatment or prevention of disorders mediated by COX-2 and carbonic anhydrases by administering to a subject an interested compound of Formula I, or a pharmaceutically acceptable salt or composition thereof, compared to the therapeutic efficacy by either COX-2 or carbonic anhydrases alone:

wherein,
X is selected from fluoro, chloro, hydrido, or methyl; and
each of R₁ to R₅, if present, is selected independently from hydrido, or halo.

In one embodiment of strong interest, a compound selected from a group of compounds designated as Group A are demonstrated to potently inhibit CAs:

wherein Group A comprises the compounds specifically listed below:
5-{4-(aminosulfonyl)phenyl}-2,2-dimethyl-4-(3-fluorophenyl)-3(2H)furanone;
5-{4-(aminosulfonyl)phenyl}-4-(2,5-difluorophenyl)-2,2-dimethyl-3(2H)furanone;
5-{4-(aminosulfonyl)phenyl}-4-(3-chlorophenyl)-2,2-dimethyl-3(2H)furanone;
5-{4-(aminosulfonyl)-3-fluorophenyl}-2,2-dimethyl-4-(3-fluorophenyl)-3(2H)furanone;
5-{4-(aminosulfonyl)-3-fluorophenyl}-4-(2,5-difluorophenyl)-2,2-dimethyl-3(2H)furanone;
5-{4-(aminosulfonyl)-3-fluorophenyl}-4-(3,4-difluorophenyl)-2,2-dimethyl-3(2H)furanone;
5-{4-(aminosulfonyl)-3-fluorophenyl}-4-(3,5-difluorophenyl)-2,2-dimethyl-3(2H)furanone;
5-{4-(aminosulfonyl)-2-fluorophenyl}-2,2-dimethyl-4-(4-fluorophenyl)-3(2H)furanone;
5-{4-(aminosulfonyl)-2-fluorophenyl}-4-(3,5-difluorophenyl)-2,2-dimethyl-3(2H)furanone;
5-{4-(aminosulfonyl)-3-methylphenyl}-2,2-dimethyl-4-(3-fluorophenyl)-3(2H)furanone; and
5-{4-(aminosulfonyl)-3-chlorophenyl}-2,2-dimethyl-4-(3-fluorophenyl)-3(2H)furanone.

In another embodiment of strong interest, a method is provided to reduce the toxicity associated with COX-2 inhibition in the treatment or prevention of COX-2 mediated disorders through COX-2 inhibition by administering to a subject a therapeutically relevant amount of a specific compound of Formula I, or a pharmaceutically acceptable salt or composition thereof:

wherein the specific compound is selected from Group A as defined above.

In yet another embodiment of strong interest, a method is provided to improve the therapeutic efficacy in the treatment or prevention of disorders mediated by COX-2 and carbonic anhydrases by administering to subject a specific compound of Formula I, a pharmaceutically acceptable salt or composition thereof, compared to the therapeutic efficacy by either COX-2 or carbonic anhydrases alone:

wherein the specific compound is selected from Group A as defined above.

The above used terms and abbreviations are defined and illustrated in Table 1.

TABLE 1
Definition of the terms and abbreviations used in the present invention.

| Term/Abbreviation | Definition & Illustration |
|---|---|
| COX | Cyclooxygenase. Examples are COX-1 (cyclooxygenase-1) |
| | and COX-2 (cyclooxygenase-2). |
| CA | Carbonic anhydrase. Examples are CA I (carbonic anhydrase |
| | I), CA II (carbonic anhydrase II), and the like. |
| GI | Gastrointestinal |
| Alkyl | Linear or branched alkyl radical having 1~5 carbon atom(s). |
| Lower Alkyl | Denoting an alkyl radical having 1~3 carbon atom(s). |
| Haloalkyl | Alkyl radical substituted with one or more halogen atom(s). |
| | Examples are fluoromethyl (F—CH₂—), 1-chloroethyl (CH₃—CHCl—), |
| | trifluoromethyl (CF₃—), and the like. |
| Halo | Halogen atom such as fluorine, chlorine, bromine, or iodine. |
| Hydrido | Single hydrogen atom. |
| Acyl | “—C(O)—” substituted with an alkyl radical. Examples are |
| | acetyl [CH₃—C(O)—], propionyl [CH₃CH₂—C(O)—], and the like. |
| Alkoxy | Oxy radical to which an alkyl radical is attached. Examples |
| | are methoxy, ethoxy, iso-propyloxy, and the like. |
| N-Alkylamino | “—NH—” substituted with an alkyl radical. Examples are N- |
| | methylamino (CH₃—NH—), N-ethylamino (CH₃CH₂—NH—), and |
| | the like. |
| N-Acylamino | “—NH—” substituted with an acyl radical. Examples are N- |
| | acetylamino [CH₃—C(O)—NH—], N-propionylamino [CH₃CH₂—C(O)—NH—], |
| | and the like. |
| Formyl | “CHO—” radical. |
| Methylenedioxy | “—O—CH₂—O—” radical. |

### Inhibition of Carbonic Anhydrases

Compounds of Formula I were assessed for their inhibitory activities against human CAs according to methods described in a literature and a prior art. [Anal. Biochem. vol 175, 289-297 (1988); WO 2004/014352] Details of the employed assay methods (Method A and Method B) for the inhibition of CAs are provided in the section of “MATERIALS AND METHODS” of this invention. Provided below are some of compounds of Formula I assayed for the inhibition of CAs.

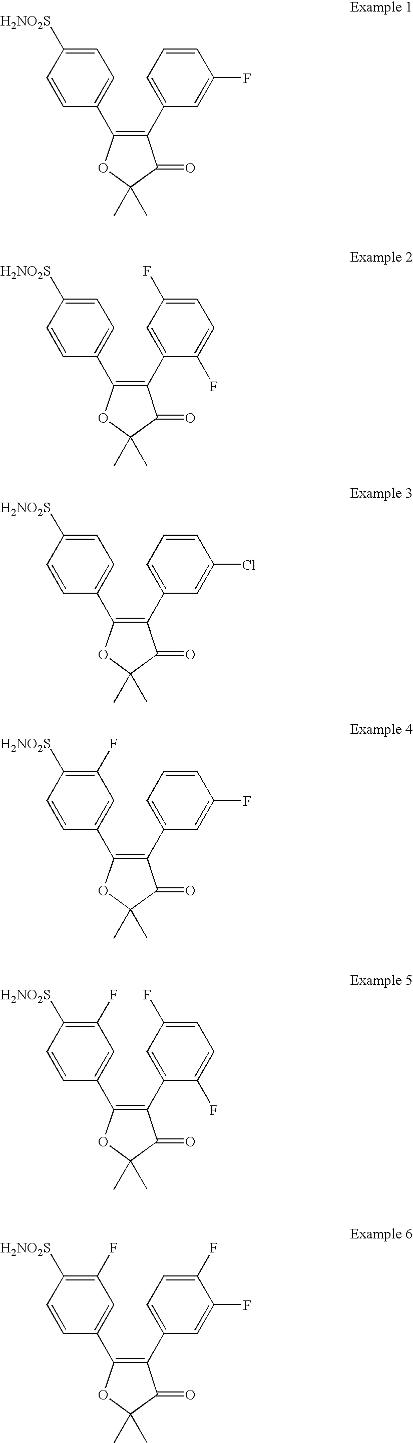

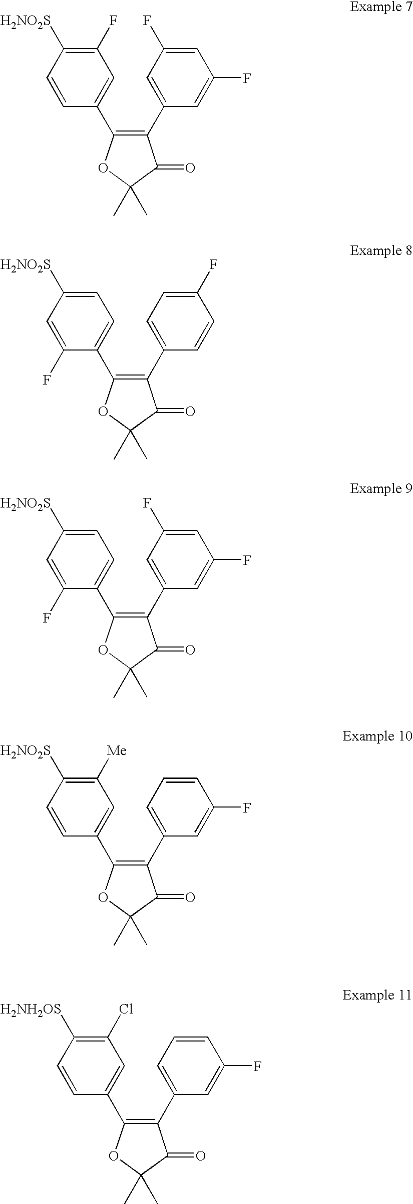

Some of the obtained CA inhibitory data are summarized in Table 2 for COX-2 inhibitors of Formula I. Acetazolamide was used as a positive comparator for the CA inhibition studies of this invention. Acetazolamide is a diuretic agent that has been in clinical use for decades.

TABLE 2
Observed IC₅₀'s for the inhibition of CA I and CA II.

| | |
|---|---|
| IC₅₀ by | IC₅₀ by |
| Method A, μM | Method B, μM |

| Compound | CA I | CA II | CA I | CA II |
|---|---|---|---|---|
| Acetazolamide | 9.0 | 0.40 | 0.63 | 0.016 |
| | | | (0.25)¹ | (0.012)¹ |
| | | | | (0.030)² |
| Example 1 | 3.0 | 0.80 | 0.29 | 0.063 |
| Example 2 | 3.2 | 0.74 | | |
| Example 3 | 1.8 | 0.53 | | |
| Example 4 | 1.7 | 0.45 | 0.21 | 0.027 |
| Example 5 | 1.3 | 0.48 | 0.24 | 0.014 |
| Example 6 | 5.0 | 0.91 | | |
| Example 7 | 3.4 | 0.63 | | |
| Example 8 | 1.9 | 0.42 | | |
| Example 9 | 4.7 | 0.63 | | |
| Example 10 | 9.1 | 5.4 | | |
| Example 11 | 8.1 | 2.9 | | |

¹Literature values cited from J. Med. Chem. vol 47, 550-557 (2004).
²Literature value cited from WO 03/013655.

Safety Benefits from Partial Inhibition of CA by a COX-2 Inhibitor

CA is abundantly present in the epithelial layer of the GI tract. The stomach and colon show very high CA activity. Whilst the jejunum contains considerable amounts of CA I and CA II, the ileum is enriched with smaller amounts of CAs. Over 100 μM of CAs was estimated to be present in the gastric mucosa. [Expert Opin. Ther. Patents vol 10, 575-600 (2000)] Table 3 summarizes the enrichments of CAs in various organs of dog. [Physiol. Rev. vol 47, 595-781 (1967)] It has to be noted that the tissue abundance profile of CAs in humans may be somewhat different from that in dogs. For example, an enrichment of 140 μM was observed in the human erythrocytes, which was significantly higher than the corresponding enrichment in Table 3 in dogs. [Am. J. Physiol. vol 181, 149-156 (1955)]

Inhibition of CA in the GI tract could show a multitude of physiological implications Inhibition of CA in the gastric mucosa could suppress the acid secretion or increase bicarbonate (HCO₃⁻) secretion, by which the gastric mucosa would be protected from the tissue damage by the gastric acid. However, overt inhibition of CAs in the upper GI tract could cause GI disturbance from improper pH homeostasis in the upper GI tract. Thus, partial inhibition of CAs in the upper GI tract would be useful to improve the GI safety of a COX-2 inhibitor without incurring adverse events from overt inhibition of CAs in the GI tract.

TABLE 3
Observed enrichments of CAs in dogs. [Physiol. Rev. vol 47,
595-781 (1967)]

| Organ | Sub-Structure | Enrichment, μM |
|---|---|---|
| Erythrocyte (RBC) | | 24-40 |
| Kidney | Cortex | 8-11 |
| | Medulla | 0-1 |
| Eye | Lens | 5-8.5 |
| | Retina | 6.8 |
| Stomach | Parietal Cell | 136 |
| Pancreas | | 0.34 |
| Prostate | | 0 |
| Salivary Glands | Parotid | 22 |
| | Submaxillary | 3 |
| | Sublingual | 0.88 |
| Brain | Choroid Plexus | 6-8 |
| Liver | | 0.34 |

CA is abundantly present also in the kidney (mostly in the cortex region) at an estimated total concentration of 8˜11 μM. Inhibition of the CAs in the kidney decreases the sodium reuptake in the renal tubules, which is the basis for the diuretic use of currently available CA inhibitors. Since overt inhibition of CAs in the kidney could result in metabolic acidosis and electrolyte imbalance, partial inhibition of CAs in the kidney may be desired to reduce frequently observed renal adverse events of edema, sodium retention, and hypertension associated with the use of a COX-2 inhibitor especially in some elderly people relying much on the renal COX-2 for their renal function. A dual inhibitor of COX-2 and CAs would show improved renal safety compared to a selective COX-2 inhibitor, as long as the renal CAs are inhibited at its therapeutic dose moderately so as not to cause the adverse events associated with overt inhibition of the renal CAs. It has to be noted that use of selective COX-2 inhibitors didn't interfere with the therapeutic effect of diuretic drugs in human subjects, [Am. J. Cardiol. vol 90, 959-963 (2002)] implying that the COX-2 inhibition in the kidney might be compatible with diuresis by partial inhibition of renal CAs.

Normally, erythrocytes constitute ˜40% of whole blood by volume in human subjects. It is often the case that erythrocytes act as a huge reservoir for a drug inhibiting CAs due to the abundant presence of CAs in the erythrocyte. Strong affinity of a drug for erythrocytes may lead to a significant reduction in the plasma concentration of the drug from what would be expected by its physicochemical properties such as the lipophilicity and plasma protein binding. Given that plasma is in direct contact with the endothelial layer, a reduced plasma concentration of a COX-2 inhibitor would mean an attenuated COX-2 inhibition in the endothelial layer. Since inhibition of the endothelial COX-2 has been implicated to increase the risk of thromboembolic events, [Science vol 296, 539-541 (2002)] a reduction in the plasma concentration of a COX-2 inhibitor due to a strong uptake by erythrocytes is somehow translated into an improvement in the cardiovascular safety of the COX-2 inhibitor.

Overt inhibition of CAs could result in unwanted side effects including GI disturbance, metabolic acidosis, and electrolyte imbalance. A moderate extent of CA inhibition, however, would be useful in obtaining a meaningful extent of beneficial physiological effects in the GI tract, kidney and systemic circulation. Therefore, improved GI, cardiovascular and renal safety profiles would be expected for a potent COX-2 inhibitor showing a moderate (not overt) level of CA inhibition at therapeutic dose for the treatment or prevention of COX-2 mediated disorders, when compared to a COX-2 inhibitor without a significant CA inhibitory activity.

Compounds of Formula I are potent COX-2 inhibitors as disclosed in prior art WO 00/61571. For example, an ED₅₀ of 0.1 mg/kg/day, bid was observed when Example 1 was orally administered to male rats for the treatment of adjuvant-induced arthritis. [J. Med. Chem. vol 47, 792-804 (2004)] Due to their abundant presence, CAs are hardly fully occupied by administration of a carbonic anhydrase inhibitor at small doses regardless of its CA inhibitory potencies. Even though compounds of Formula I inhibit CAs with potencies comparable to those of acetazolamide, the inhibitory extent of CAs by those compounds are expected to be small in tissues of safety concern due to their small therapeutic dose for the treatment or prevention of COX-2 mediated disorders. CAs are present in the kidney and the upper GI tract in large quantities, and therefore hard to be saturated (i.e. fully occupied) by a dual inhibitor of COX-2 and CAs at a small therapeutic dose.

### Attenuation of COX-2 Inhibition by Strong Affinity for CA

Inhibition of CAs by a COX-2 inhibitor would show safety benefits in the GI tract and the kidney for the treatment or prevention of disorders mediated through COX-2 for a different reason. If CAs are present in a large excess of a COX-2 inhibitor with strong affinities for CAs, the free cytosolic concentration of the COX-2 inhibitor would be attenuated as much as its affinities for CAs dictate. Since COX-2 is a membrane-bound enzyme in equilibrium with the cytosol across an intracelluar membrane, [J. Biol. Chem. vol 270, 10902-10908 (1995)] a notable reduction in the free cytosolic concentration of the COX-2 inhibitor should lead to a significant attenuation in the extent of COX-2 inhibition. Overt inhibition of COX-2 is associated with adverse events in the GI tract and the kidney, which are highly enriched with CAs. The COX-2 inhibitory extent by a highly potent COX-2 inhibitor with strong affinities for CAs should be much smaller in the upper GI tract and the kidney than in other tissues of therapeutic concern at therapeutic dose for the treatment or prevention of COX-2 mediated disorders. For a COX-2 inhibitor with a small therapeutic dose and strong affinities for CAs, the inhibitory extents of CAs in the upper GI tract and the kidney tend to be not significant at its therapeutic dose despite its strong affinity for CAs. Consequently therapeutic use of the COX-2 inhibitor is unlikely to cause overt inhibition of the CAs in the upper GI tract and the kidney, which excludes the possibility of adverse effects from overt inhibition of CAs.

In theory, the free cytosolic concentration of a COX-2 inhibitor binding to CA with an affinity can be calculated in the presence of a large excess amount of CA. Equation (1) describes the binding equilibrium between a COX-2 inhibitor and CA. The inhibition constant K_{I} can be approximated as in equation (2), if CA is present in a large excess of the total concentration of the COX-2 inhibitor.

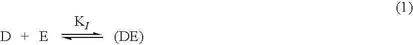

wherein,

D and E are COX-2 inhibitor and carbonic anhydrase, respectively,

and, (DE) is the complex between D and E.

K_{I}=[D][E]/[(DE)]=(E₀−[(DE)])[D]/[(DE)]≃E₀[D]/[(DE)] (2)

wherein,

[D] and [E] are free (uncomplexed) concentrations of D and E, respectively,

E₀ is the total concentration of CA,

D₀ is the total concentration of the COX-2 inhibitor, i.e. D₀=[D]+[(DE)],

and, E₀>>D₀

The ratio between the unbound (free) and bound (complexed) COX-2 inhibitor can be calculated for a variety of binding affinities between the COX-2 inhibitor and CA. The following simulation cases are provided to explain how significantly the free cytosolic concentration of a potent COX-2 inhibitor is attenuated by its strong affinity for CA, given a reasonable range of CA enrichments in the kidney, upper GI tract, or erythrocytes.

[Simulation Case 1]: Assuming that CA is present at 100 μM similarly as in the stomach, and that a potent COX-2 inhibitor binds to CA with a K_{I} of 100 nM, the ratio between the free and the bound (complexed) concentration of the COX-2 inhibitor may be calculated according to equation (3). Thus, about 99.9% of the drug molecules are present as bound to CA in the cytosolic solution, illustrating well how significantly the free drug concentration can be attenuated by strong binding to CA in the presence of CA in large excess of the drug. In this simulation, CA is in a large excess of the drug, implying that most of CA remains unbound and consequently that the physiological functions of CA remain undisturbed despite the strong affinity of the drug for CA.

[D]/[(DE)]≃Kᵢ/E⁰=(100 nM)/(100 μM)=10⁻³ (3)

[Simulation Case 2]: Assuming that CA is present at 10 μM similarly as in the renal cortex, and that a potent COX-2 inhibitor binds to CA with a K_{I} of 100 nM, the ratio between the free and the bound (complexed) concentration of the COX-2 inhibitor may be calculated according to equation (4). Thus, about 99% of the drug molecules are present as bound to CA in the cytosolic solution, illustrating well how significantly the free drug concentration can be attenuated by strong binding to CA in the presence of CA in a large excess of the drug.

[D]/[(DE)]≃K_{I}/E₀=(100 nM)/(10 μM)=10⁻² (4)

[Simulation Case 3] Assuming that CA is present at 10 μM similarly as in the renal cortex, and that a potent COX-2 inhibitor binds to CA with a K_{I} of 1 μM, the ratio between the free and the bound (complexed) concentration of the COX-2 inhibitor may be calculated according to equation (5). Thus, about 90% of the drug molecules are present as bound to CA in the cytosolic solution, illustrating well how significantly the free drug concentration can be attenuated by strong binding to CA in the presence of CA in a large excess of the drug.

[D]/[(DE)]≃K_{I}/E₀=(1 μM)/(10 μM)=0.1 (5)

Above simulation cases indicate that over 90% of the COX-2 inhibitor in the cytosol is present as bound (complexed) to CA, if CA is enriched at over 10 μM and the COX-2 inhibitor binds to CA with a K_{I} smaller than 1 μM. As exemplified in Table 2, compounds of Formula I show strong affinities for CAs and a K_{I} of 1 μM to 100 nM would not be an unrealistic assumption for a COX-2 inhibitor of Formula I. Therefore, strong binding to CA may result in a significant attenuation in the free cytosolic concentration and the COX-2 inhibitory activity in tissues of safety concern, including the upper GI tract, the kidney, and so on.

As discussed previously, COX-2 inhibitors may not be completely free of the toxicity in the GI tract and the kidney. Such toxicity is believed to originate partly from the inhibition of COX-2 in the GI tract and the kidney. It is often the case that COX-2 inhibitors are associated with the GI and renal toxicity in a dose-dependent manner, suggesting partial inhibition of COX-2 or COX-1/COX-2 in such organs of safety concern at therapeutic dose. CAs are abundantly present in the GI tract and the kidney. A COX-2 inhibitor with a strong affinity for CA tends to show an attenuated COX-2 inhibitory activity in the GI tract and the kidney, which may lead to reduced toxicity from the COX-2 inhibition in such organs. Thus, potent inhibition of CA by a COX-2 inhibitor with a strong COX-2 inhibitory potency reduces or prevents the toxicity associated with the inhibition of COX-2.

Erythrocytes (red blood cells) constitute a major portion of the whole blood. The erythrocyte contains a huge amount of CAs (see Table 3). Thus, a COX-2 inhibitor with a strong affinity for CA tends to distribute preferentially to the erythrocytes over the plasma. Therefore, the drug concentration in the plasma tends to be small compared to that in the whole blood. The effect of such a small plasma level would be translated into a small drug level in the endothelial layer, an organ in direct contact with the blood where COX-2 would be working to produce prostacyclin. The end effect of the strong affinity of the COX-2 inhibitor would be a reduced inhibition of the prostacyclin synthesis in the endothelial layer. Prostacyclin is a vasodilatory prostaglandin inhibiting the platelet activity. [Science vol 296, 539-541 (2002)] Reduced inhibition of the prostacyclin synthesis would contribute to the improvement in the cardiovascular safety for a COX-2 inhibitor with strong affinities for CAs in the treatment or prevention of COX-2 mediated disorders, compared to other COX-2 inhibitors without CA inhibitory activity.

Therapeutic Benefits from Partial Inhibition of CA

Therapeutic scope of COX-2 inhibitors would be much broader than commonly practiced indications which include osteoarthritis, rheumatoid arthritis, post operative pains, migraines, menstrual pains, and so on Inhibition of COX-2 has been implicated to be useful for the treatment or prevention of some types cancer, which include colorectal and breast cancers. [Cancer Det. Prev. vol 28, 127-142 (2004)] COX-2 inhibitors would be useful to treat ocular diseases involving inflammation and angiogenesis. [Trends Mol. Med. vol 9, 73-78 (2003)] COX-2 inhibitors would be useful to treat mild to moderate migraines. COX-2 inhibitors would show disease modifying activity against osteoclastogenesis. [Pain vol 107, 33-40 (2004)]

CA inhibitors have been known to be useful to treat a variety of disorders overlapping with those manageable with COX-2 inhibitors. For example, CA inhibitors would be useful to treat or manage glaucoma, genetic hemiplegic migraine, osteoporosis and some types of tumors. [Expert Opin. Ther. Patents vol 10, 575-600 (2000)]

A compound of Formula I would be useful to treat a variety of COX-2 mediated disorders at relatively small therapeutic dose. Even though its therapeutic dose would be small to inhibit CA(s) partially and not enough to induce a meaningful level of therapeutic effect through CA inhibition alone in tissues of therapeutic concern for COX-2 mediated disorders, such partial inhibition of CA(s) would still be of therapeutic significance, if combined with COX-2 inhibition to produce additional therapeutic effect in some types of COX-2 mediated disorders, including but not limited to, glaucoma, migraine, osteoporosis, and certain types of cancers.

### Plasma Level of Therapeutic Relevance

Compounds of Formula I are highly potent COX-2 inhibitors and therefore expected to produce therapeutic effect at small systemic exposure for the treatment or prevention of COX-2 mediated disorders. For example, an ED₅₀ of 0.1 mg/kg/day, bid (i.e. dosed two times per day) was observed when Example 1 was orally administered to treat adjuvant-induced arthritis in male Lewis rats. [J. Med. Chem. vol 47, 792-804 (2004)] Therefore the plasma concentration of Example 1 for Cₘₐₓ for the ED₅₀ would be of therapeutic relevance for the treatment of arthritis and arthritis associated disorders. Oral administration of Example 1 at 1 mg/kg was associated with a plasma Cₘₐₓ value of 82 ng/ml in male SD rats. A Cₘₐₓ for 0.1 mg/kg/day, bid (i.e. 0.05 mg/kg) would be calculated to be 4.1 ng/ml (i.e. 82 ng/ml divided by 20). Example 1 would show therapeutic effect at plasma concentrations of a few ng/ml, which were far lower than the enrichments of CAs in the upper GI tract and the kidney. Therefore, CAs in the upper GI tract and the kidney would not be inhibited much at therapeutic dose for the treatment of COX-2 mediated disorders, in spite of the strong affinities of Example 1 for CAs.

### Distribution Profile Reflecting Strong Binding to CAs

CAs are highly enriched in the erythrocyte. A total of over 100 μM of CAs is known to be present in the human erythrocyte (see Table 3). A CA inhibitor tends to preferentially distribute to the erythrocytes over the plasma in whole blood. For example, valdecoxib was known to preferentially distribute to the erythrocytes with a ratio of 2.5:1 over the plasma despite its strong plasma protein binding of 98% in human whole blood, [Pharmacology Review (Valdecoxib), FDA Application # 21-341, p 217] whereas there was no noticeable preferential distribution with rofecoxib, a COX-2 inhibitor with a plasma protein binding of 87%. [Pharmacology Review (Rofecoxib), FDA Application # 21,042, p 23]

Compounds of Formula I bind strongly to the plasma protein. For example, Example 1 showed a plasma protein binding over 99% in a pooled rat plasma at 1 μg/ml in one binding assay. Example 1 showed preferential distribution to the erythrocytes over the plasma with ratios of 31:1 and 45:1 at 8.2 μg/ml and 0.8 μg/ml Example 1 in the rat whole blood, respectively (see MATERIALS AND METHODS for experimental details). Example 1 was found to preferentially distribute to the erythrocytes with a ratio of 13:1 at 13.2 μg/ml Example 1 in human whole blood.

When a ¹⁴C-labeled compound of Example 1 was used to determine the plasma protein binding and preferential distribution in the erythrocytes, plasma protein bindings of ca 90% were observed for a broad range of concentrations of Example 1. Example 1 was also found to even more preferentially distribute to erythrocytes over plasma. It needs to be noted that use of a radio-labeled compound is a more sensitive analytical approach than use of the non-labeled compound for assessing drug distribution profiles in plasma and whole blood. Therefore, the strong CA binding affinity of Example 1 is well reflected in its preferential distribution to the erythrocytes despite its strong plasma protein binding.

### Improved Safety Reflecting Strong Binding to CAs

In rats, the jejunum and ileum were the primary target sites of the GI adverse events following repeated oral administrations of rofecoxib and celecoxib. Similar situations were observed in repeat oral dosing studies with valdecoxib. [Pharmacology Review (Rofecoxib), US FDA Application #21-042, pp 72-92; Pharmacology Review (Celecoxib), US FDA Application #20-998, pp 10-42; Pharmacology Review (Valdecoxib), US FDA Application #21-341, pp 27-54] Reflecting their high COX-2 selectivities over COX-1, celecoxib, rofecoxib and valdecoxib were not associated with gastric toxicity in rats at doses, where the intestinal toxicity began to come out. Even though selective COX-2 inhibitors are considered to possess better GI safety than traditional NSAIDs, long term GI safety margins (i.e. the ratio between human therapeutic exposure and NOAEL exposure for the GI safety in animals) were found to be a matter of only a few fold for selective COX-2 inhibitors including rofecoxib, celecoxib and valdecoxib.

Not all selective COX-2 inhibitors would show the same pattern of GI toxicity. Meloxicam is a selective COX-2 inhibitor with a modest COX-2 selectivity over COX-1 (13-fold selectivity in human whole blood). Repeat dosing of meloxicam in rats resulted in adverse findings in the stomach and the intestines. However, gastric findings such as peptic pyloric ulcers were observed at lower dose of meloxicam than intestinal findings such as duodenal perforations with peritonitis. [Pharmacology Review (Meloxicam), US FDA Application #20-938, pp 25-43] Such observation could be explained by a significant extent of COX-1 inhibition in the stomach, paralleling with the GI toxicity pattern of traditional NSAIDs. [Gut vol 49, 443-453 (2001)] The duodenum was the primary target organ among the intestines following repeat dosing of meloxicam in rats.

The GI toxicity of Example 1 was evaluated by orally administering Example 1 to rats or monkeys for up to 4 weeks at various daily dose levels. Intestinal adverse effects (primarily in the caecum) were dose limiting in rats. However, there were no positive histopathological findings in the stomach in rats even at a daily systemic exposure of ca 30,000 hr×ng/ml following repeated oral administrations of Example 1 to rats for 4 weeks. Considering that Example 1 was conceived to possess significant anti-inflammatory activities at plasma concentrations of a few ng/ml or so by adjuvant-induced arthritis in rats, a daily systemic exposure of ca 30,000 hr×ng/ml is taken as a huge excess of a therapeutically relevant exposure. Furthermore, the caecum was the primary target site of the intestinal adverse findings, which was different from the cases of known COX-2 inhibitors with a good COX-2 selectivity over COX-1.

Repeated oral administrations of Example 1 up to 48 mg/kg/day were well tolerated in monkeys. Repeated oral administrations of Example 1 in monkeys for 4 weeks were not associated with positive histopathological findings in the intestines. Repeated administrations of Example 1 for 4 weeks were associated with one case of focal mucosal degeneration in the stomach. However, the subject (one out of 18 subjects treated with Example 1 for 4 weeks) with the histopathological finding in the stomach showed a daily systemic exposure larger than 20,000 hr×ng/ml on the final day of the treatment. Monkeys showed robust gastric tolerance in the 4 week repeat dose study with Example 1. Therefore, the strong CA binding affinity of Example 1 is partly reflected in its improved gastric safety margin at least in experimental animals.

### Materials and Methods

Materials: COX-2 inhibitors of Formula I used in this invention were prepared as disclosed in the prior art WO00/61571. Human CA I (Sigma Cat # C-6165) and CA II (Sigma Cat # C-4936) were purchased from Sigma and used without further purification. Acetazolamide (Sigma Cat # A-6011) was obtained from Aldrich. Phenol red (Aldrich Cat # 11452-9) and p-nitrophenylacetate (Sigma Cat # N-8130) were purchased from Aldrich and Sigma, respectively, and used without further purification. DMSO and buffer agents were obtained from Sigma. Distilled water was prepared in-house by distilling deionized water.

Inhibition Assay for CA (Method A): CA inhibitory activities by COX-2 inhibitors of this invention were assessed by a previously reported method. [Anal. Biochem. vol 175, 289-297 (1988)] The employed method is summarized as follows: To a mixed solution of 400 μl phenol red solution (12.5 mg/l phenolsulfonephthalein and 2.6 mM NaHCO₃ in distilled water) and 300 μl of 19.5 nM CA in 0.17% aqueous DMSO containing a designated amount of an inhibitor, was continuously bubbled CO₂ until the color of the solution turned yellow. Then the reaction of CA was initiated by adding 100 μl of a carbonate buffer solution (0.3 M Na₂CO₃ and 0.206 M NaHCO₃ in distilled water). Then the interval was measured from the initiation of the reaction to the point of the color change of the solution from purple to yellow. During the entire reaction, the reaction solution was kept bubbled with CO₂. The interval measured for a solution without an inhibitor was used as the control value for the 100% CA reaction, which was used to calculate the % inhibition value for a designated concentration of inhibitor according to equation (6).

% Inhibition=[1−(Interval of Color Change with Inhibitor)/(Interval of Color Change without Inhibitor)]×100 (6)

Inhibition Assay for CA (Method B): The inhibitory activities of CA by inhibitors were assessed alternatively by a method described in prior art WO03/013655. The employed method is briefly described as follows. An aqueous reaction mixture consisting of 2 Wilbur-Anderson units of CA, 4 mM p-nitrophenylacetate, 5% DMSO, 0.1 M Na₂SO₄, and 50 mM Tris-HCl buffer at pH 7.6 was prepared to contain a designated amount of inhibitor. The esterase activity of CA (the hydrolysis of p-nitrophenylacetate) was followed by the increases in absorbance at 405 nm using an ELISA reader at room temperature. The initial rate (slope of the initial absorbance data) data from the absorbance data were used to calculate % inhibition of CA at a designated concentration of inhibitor according to equation (7).

% Inhibition=[1−(Initial Rate with Inhibitor)/(Initial Rate without Inhibitor)]×100 (7)

Partitioning of Compound between the Plasma and the Erythrocytes: 3 μl of a DMSO stock solution containing a compound of Formula I was added to 300 μl of blood in an eppendorf tube, which was collected from a male SD rat in a tube containing ACD (acid, citrate, and dextrose) as anticoagulant. Then the blood solution was vortexed for seconds and incubated for 30 min at 37° C. on a table-top incubator, which was followed by sedimentation by centrifugation at 3000 rpm (ca 300 g) for 10 min. The plasma layer was collected and subjected to quantification for the compound dissolved in the plasma by HPLC analysis with a pre-established calibration curve. Assuming that the erythrocytes constitute 44% of the whole blood by volume, the ratio between the compound concentrations in the erythrocytes and in the plasma could be indirectly determined according to equation (8).

$$Partitioning Ratio between Erythrocytes and Plasma = \frac{[ Concentration in Erythrocytes ]}{[ Concentration in Plasma ]} = \frac{[ \frac{( {C}_{WB} - {C}_{P} \times 0.56 )}{0.44} ]}{{C}_{P}} \qquad ( 8 )$$

wherein

C_{WB} is the concentration of compound in the whole blood, and

C_{P} is the plasma concentration of compound determined by HPLC analysis.

Alternatively, partitioning between plasma and erythrocytes was determined by using a ¹⁴C-labeled compound of Formula I and whole blood withdrawn as heparinized. Scintillation counting was used for analysis in place of HPLC.

Plasma Protein Binding of Compound: Plasma protein binding was determined for a compound of Formula I by a filtration method. An aliquot of a compound stock solution in DMSO was added to a designated volume of freshly prepared pooled plasma from male SD rats. The plasma solution was then subjected to filtration using a centrifuge filter (Amicon YM-30) at 4000 g for 30 min. The filtrate was analyzed by HPLC to determine the concentration of the compound.

Alternatively, plasma protein binding was determined by a filtration method employing a ¹⁴C-labeled compound of Formula I. Scintillation counting was used for analysis in place of HPLC.

Pharmacokinetic Studies in Rats: An appropriate amount of a compound suspended in 1% aqueous methylcelluose solution was administered to male SD rats by oral gavage. Blood samples were collected from the retro-orbital sinus at designated time points over 0 to 24 hours post dose. Plasma was separated from each withdrawn blood sample by centrifugation and the plasma sample was stored at 4° C. until HPLC analysis for the quantification of the compound. The plasma samples were analyzed by reverse phase HPLC using an appropriate internal standard and a pre-established calibration curve.

Repeat Dose Toxicity Studies: Cynomolgus monkeys or rats were orally administered on a daily basis with a compound of Formula I at 0˜48 mg/kg body weight per day for up to 4 weeks. Toxicokinetic studies were performed with treatment groups in monkeys. Satellite toxicokinetic groups were used for the toxicokinetic studies of treatment groups in rats. Animals at terminal sacrifice were evaluated for histopathological findings in the GI tract and other organs.

What is claimed is:

1. A method to treat or prevent disorders associated with carbonic anhydrases by administering to a subject a therapeutically effective amount of a compound of Formula I, or a pharmaceutically acceptable salt or composition thereof: 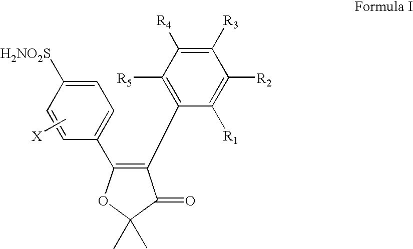
wherein,
X is selected from halo, hydrido, or lower alkyl; and
each of R₁ to R₅, if present, is selected independently from hydrido, halo, alkyl, haloalkyl, acyl, alkoxy, hydroxy, nitro, amino, N-alkylamino, N-acylamino, cyano, formyl, or azido; or two adjacent groups of R₁ to R₅ form, taken together, methylenedioxy.

2. The method of claim 1, wherein X is selected from fluoro, chloro, hydrido, or methyl; and each of R₁ to R₅, if present, is selected independently from hydrido or halo.

3. The method of claim 1, wherein the compound of Formula I is selected from the group consisting of:
5-{4-(aminosulfonyl)phenyl}-2,2-dimethyl-4-(3-fluorophenyl)-3(2H)furanone;
5-{4-(aminosulfonyl)phenyl}-4-(2,5-difluorophenyl)-2,2-dimethyl-3(2H)furanone;
5-{4-(aminosulfonyl)phenyl}-4-(3-chlorophenyl)-2,2-dimethyl-3 (2H)furanone;
5-{4-(aminosulfonyl)-3-fluorophenyl}-2,2-dimethyl-4-(3-fluorophenyl)-3(2H)furanone;
5-{4-(aminosulfonyl)-3-fluorophenyl}-4-(2,5-difluorophenyl)-2,2-dimethyl-3(2H)furanone;
5-{4-(aminosulfonyl)-3-fluorophenyl}-4-(3,4-difluorophenyl)-2,2-dimethyl-3(2H)furanone;
5-{4-(aminosulfonyl)-3-fluorophenyl}-4-(3,5-difluorophenyl)-2,2-dimethyl-3(2H)furanone;
5-{4-(aminosulfonyl)-2-fluorophenyl}-2,2-dimethyl-4-(4-fluorophenyl)-3(2H)furanone;
5-{4-(aminosulfonyl)-2-fluorophenyl}-4-(3,5-difluorophenyl)-2,2-dimethyl-3(2H)furanone;
5-{4-(aminosulfonyl)-3-methylphenyl}-2,2-dimethyl-4-(3-fluorophenyl)-3(2H)furanone; and
5-{4-(aminosulfonyl)-3-chlorophenyl}-2,2-dimethyl-4-(3-fluorophenyl)-3(2H)furanone.

4. A method to improve the therapeutic efficacy in the treatment or prevention of disorders mediated by COX-2 and carbonic anhydrases by administering to a subject a compound of Formula I, or a pharmaceutically acceptable salt or composition thereof, compared to the therapeutic efficacy by inhibition of either COX-2 or carbonic anhydrases alone: 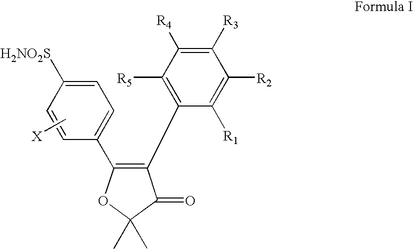
wherein,
X is selected from halo, hydrido, or lower alkyl; and
each of R₁ to R₅, if present, is selected independently from hydrido, halo, alkyl, haloalkyl, acyl, alkoxy, hydroxy, nitro, amino, N-alkylamino, N-acylamino, cyano, formyl, or azido; or two adjacent groups of R₁ to R₅ form, taken together, methylenedioxy.

5. The method of claim 4, wherein X is selected from fluoro, chloro, hydrido, or methyl; and each of R₁ to R₅, if present, is selected independently from hydrido or halo

6. The method of claim 4, wherein the compound of Formula I is selected from the group consisting of:
5-{4-(aminosulfonyl)phenyl}-2,2-dimethyl-4-(3-fluorophenyl)-3(2H)furanone;
5-{4-(aminosulfonyl)phenyl}-4-(2,5-difluorophenyl)-2,2-dimethyl-3(2H)furanone;
5-{4-(aminosulfonyl)phenyl}-4-(3-chlorophenyl)-2,2-dimethyl-3(2H)furanone;
5-{4-(aminosulfonyl)-3-fluorophenyl}-2,2-dimethyl-4-(3-fluorophenyl)-3(2H)furanone;
5-{4-(aminosulfonyl)-3-fluorophenyl}-4-(2,5-difluorophenyl)-2,2-dimethyl-3(2H)furanone;
5-{4-(aminosulfonyl)-3-fluorophenyl}-4-(3,4-difluorophenyl)-2,2-dimethyl-3(2H)furanone;
5-{4-(aminosulfonyl)-3-fluorophenyl}-4-(3,5-difluorophenyl)-2,2-dimethyl-3(2H)furanone;
5-{4-(aminosulfonyl)-2-fluorophenyl}-2,2-dimethyl-4-(4-fluorophenyl)-3(2H)furanone;
5-{4-(aminosulfonyl)-2-fluorophenyl}-4-(3,5-difluorophenyl)-2,2-dimethyl-3(2H)furanone;
5-{4-(aminosulfonyl)-3-methylphenyl}-2,2-dimethyl-4-(3-fluorophenyl)-3(2H)furanone; and
5-{4-(aminosulfonyl)-3-chlorophenyl}-2,2-dimethyl-4-(3-fluorophenyl)-3(2H)furanone.
